(19) United States
(12) Patent Application Publication
(10) Pub. No.: US 2025/0103808 A1
(43) Pub. Date: Mar. 27, 2025

(54) JOINT EXTRACTION SYSTEM AND METHOD FOR ENTITY RELATIONSHIP IN FIELD OF TRADITIONAL TIBETAN MEDICINES

(71) Applicant: Xizang University, Lhasa (CN)

(72) Inventors: Cuo YONG, Lhasa (CN); Tao YU, Lhasa (CN); ZhaXi NIMA, Lhasa (CN)

(73) Assignee: Xizang University, Lhasa (CN)

(21) Appl. No.: 18/972,491

(22) Filed: Dec. 6, 2024

Related U.S. Application Data
(63) Continuation of application No. PCT/CN2024/097796, filed on Jun. 6, 2024.

(30) Foreign Application Priority Data
Jun. 9, 2023 (CN) 2023107097740

(51) Int. Cl. G06F 40/279; G06F 40/40; G06N 3/0442; G06N 3/047; G06N 3/09

(52) U.S. Cl. CPC G06F 40/279; G06F 40/40; G06N 3/0442; G06N 3/047; G06N 3/09

(57) ABSTRACT

The present invention proposes a joint extraction system and method for an entity relationship in the field of traditional Tibetan medicines, and relates to the field of artificial intelligence. The joint extraction method for an entity relationship in the field of traditional Tibetan medicines includes acquiring training samples; converting the training samples into word vectors; classifying the training samples, and fusing a classifying result with the word vectors to obtain static fusion features; constructing a binary dynamic model, and feeding the static fusion features into the binary dynamic model to obtain a final predicted tag sequence; calculating a loss value of the binary dynamic model, and updating parameters to obtain an updated binary dynamic model; and performing joint extraction of an entity relationship by using the updated binary dynamic model. According to the present invention, nested entities can be labeled, and prediction accuracy can be improved; and entity boundaries are enhanced.

### CROSS-REFERENCE TO RELATED APPLICATIONS

The application claims priority to Chinese patent application No. 2023107097740, filed on Jun. 9, 2023, the entire contents of which are incorporated herein by reference.

### TECHNICAL FIELD

The present invention relates to the field of artificial intelligence, in particular to a joint extraction system and method for an entity relationship in the field of traditional Tibetan medicines.

### BACKGROUND

Currently, there are two main research methods for joint extraction of an entity relationship: a pipeline method and a joint method. By the pipeline method, a joint extraction task is decomposed into entity recognition and relationship extraction, and joint extraction is achieved in a series manner. However, this manner has problems such as lack of interaction and error accumulation, which affect the effectiveness of models. Compared with the pipeline method, the joint method considers more interaction between subtasks and can directly obtain triplet data through end-to-end models, thereby improving the effectiveness of the models and gradually becoming a current hot research direction.

NovingTagging is a joint extraction model based on a sequence labeling method, which belongs to a joint decoding manner. Compared with the pipeline method, the joint method enhances the interaction between entity recognition and relationship extraction tasks, and reduces the problem of error accumulation propagation. A NovingTagging model has the following shortcomings: 1, nested entities cannot be labeled due to limitation of the sequence labeling manner; and 2, the models are single in input features and limited in learning capacity.

By a NovingTagging method, decoding can be directly performed to obtain triplet data by superimposing a unified decoder on an encoder, but a sequence labeling strategy of the NovingTagging method cannot label the nested entities, that is, short entities in long entities, and thus, the nested entities cannot be recognized. Due to limit to feature learning capability of the NovingTagging model, it is impossible to effectively learn important information in features and decode entity tags, resulting in poor joint extraction effects of the models.

### SUMMARY

The objective of the present invention is to provide a joint extraction system and method for an entity relationship in the field of traditional Tibetan medicines, which can enhance the quality of model input features, label nested entities, and improve robustness and effectiveness.

The present invention is achieved as follows:

In a first aspect, the present application provides the joint extraction system for an entity relationship in the field of traditional Tibetan medicines, including a word embedding layer, a class feature static fusion layer, and a binary dynamic model,
where the word embedding layer is used for converting inputted texts into word vectors;
the class feature static fusion layer is used for dividing the inputted texts into three classes of medicinal materials, prescriptions and diagnosis and treatment methods, and fusing the word vectors with the corresponding classes to obtain static fusion features; and
the binary dynamic model is used for acquiring dynamic features according to the static fusion features, fusing the dynamic features with the static fusion features to obtain overall fusion features, and constructing a final predicted tag sequence according to the overall fusion features.

In a second aspect, the present application provides the joint extraction method for an entity relationship in the field of traditional Tibetan medicines, including the following steps:
S1, acquiring text samples related to traditional Tibetan medicines, as training samples;
S2, converting the training samples into word vectors recorded as (bs, seq_len, dim1), where bs is a batch size, seq_len is a sentence length, and dim1 is a word vector feature dimension;
S3, classifying the training samples, and fusing a classifying result with the word vectors to obtain static fusion features;
S4, constructing a binary dynamic model, and feeding the static fusion features into the binary dynamic model to obtain a final predicted tag sequence;
S5, calculating a loss value of the binary dynamic model, and updating parameters to obtain an updated binary dynamic model; and
S6, performing joint extraction of an entity relationship using the updated binary dynamic model.

Further, the above step S3 includes the following steps:
S3-1, classifying the training samples into three classes of medicinal materials, prescriptions and the diagnosis and treatment methods, to obtain a classifying result as class features of the training samples;
S3-2, vectoring each data in the training samples according to the class features of the training samples to obtain vectored sample data recorded as (bs2, seq_len2, dim2), where bs2 is a batch size of the vectored sample data, seq_len2 is a sentence length of the vectored sample data, and dim2 is a class feature dimension of the vectored sample data; and
S3-3, fusing the vectored sample data with the word vectors to obtain static fusion features recorded as (bs3, seq_len3, dim1+dim2), where bs3 is a batch size of the static fusion features, seq_len3 is a sentence length of the static fusion features, and dim1+dim2 is a fusion feature dimension of the static fusion features.

Further, the above step S4 includes the following steps:
S4-1, feeding the static fusion features into a static feature learning module to obtain a predicted tag sequence;
S4-2, feeding the predicted tag sequence and the static fusion features into a multi-feature dynamic fusion layer to obtain overall fusion features; and
S4-3, feeding the overall fusion features into a dynamic feature learning module to obtain a final predicted tag sequence.

Further, the above step S4-1 includes the following steps:
S4-1-1, feeding the static fusion features into a BiLSTM encoding layer to obtain encoded static fusion features;
S4-1-2, processing the encoded static fusion features by using a Dropout function, to obtain processed static fusion features;
S4-1-3, mapping dimensions of the processed static fusion features to tag dimensions by using a linear classifying layer, to obtain mapped static fusion features;
S4-1-4, calculating a global optimal tag through a reward and punishment mechanism layer according to the mapped static fusion features;
S4-1-5, inputting a global optimal path into a TagScorel layer, to acquire constraint tags; and
S4-1-6, inputting the constraint tags into a CRF decoding layer, to obtain the predicted tag sequence.

Further, the dynamic feature learning module and the static feature learning module in the step S4-3 have the same structure.

Further, the above step S4-1-4 includes the following steps:
S4-1-4-1, calculating scores of each path of the training samples according to the obtained mapped static fusion features;
S4-1-4-2, obtaining a loss value loss of a CRF reward and punishment mechanism layer according to a formula:

$$P ( y \vert x ) = \frac{{e}^{S ( x , {y}^{′} )}}{{\sum }_{i}^{n} {e}^{S ( x , {y}_{i} )}}$$

$$loss = - log ( P ( y \vert x ) ) = log ( {\sum }_{i}^{n} {e}^{S ( x , {y}_{i} )} ) - S ( x , {y}^{′} )$$

where P(y|x) represents a probability that the current path is a correct path, e^{s(x,y′)} represents scores of the current correct path,

$${\sum }_{i}^{n} {e}^{S ( x , {y}_{i} )}$$

represents a sum of possible scores of the each current path, i represents an i^{th} path, n represents n paths in total, S(x,y′) represents scores obtained by correct paths, y′ represents that the current path is the correct path, and x represents an entity;
S4-1-4-3, setting the reward and punishment mechanism, performing counter propagation by using the loss value loss of the CRF decoding layer, and modifying parameters of the CRF reward and punishment mechanism layer to obtain a modified CRF reward and punishment mechanism layer; and
S4-1-4-4, calculating the global optimal path by using the modified CRF reward and punishment mechanism layer.

Further, the above step S4-2 includes the following steps:
S4-2-1, feeding the predicted tag sequence into a word segmentation information extractor, and constructing word segmentation information for an entity according to predicted tags, where the word segmentation information at a start position of the entity is labeled as 1, the word segmentation information at an end position of the entity is labeled as 3, the word segmentation information at a middle position of the entity is labeled as 2, and the word segmentation information of a non-entity is labeled as 0;
S4-2-2, feeding the predicted tag sequence into a position information extractor, and constructing position information for the entity according to tags;
S4-2-3, fusing the word segmentation information with the position information, to obtain dynamic fusion features; and
S4-2-4, fusing the dynamic fusion features with the static fusion features to obtain the overall fusion features.

Further, the loss value of the binary dynamic model is calculated in the above step S5 by using a loss value between the predicted tag sequence of the static feature learning module and the final predicted tag sequence of the dynamic feature learning module.

In a third aspect, the present application provides an electronic device, including a memory, being used for storing one or more programs; and a processor, where when the one or more programs are executed by the processor, the method according to any one in the first aspect is realized.

Compared with the prior art, the present invention at least has the following advantages or beneficial effects:
the present invention proposes a joint extraction system and method for an entity relationship in the field of traditional Tibetan medicines, through improvement on a labeling strategy, the nested entities can be labeled, and further, triplet data and the nested entities can be directly extracted through models. In a training process, class information of current instances is statically fused, and the effects of the models are improved by fusing the class features. Vector scores and a real tag result are matched, tags which are classified correctly are rewarded and tags which are wrongly correctly are punished, finally, the loss value is calculated by using the scores after processing with the reward and punishment mechanism, the value is continuously subjected to counter propagation to modify a tag classifying result of the models, and prediction accuracy is improved. Entity boundaries are enhanced by using dynamic feature fusion, such that the models can better recognize the entities and relationship.

### BRIEF DESCRIPTION OF THE DRAWINGS

To describe the technical solutions in the embodiments of the present invention more clearly, the following briefly describes the accompanying drawings required for describing the embodiments. It should be understood that, the following accompanying drawings show merely some embodiments of the present invention, and therefore should not be regarded as a limitation on the scope. A person of ordinary skill in the art may still derive other related drawings from these accompanying drawings without creative efforts.

FIG. 1 is an overall model diagram of the present invention;

FIG. 2 is a flowchart of the present invention;

FIG. 3 is a flowchart of a final predicted tag sequence obtained by the present invention; and

FIG. 4 is a schematic diagram for obtaining dynamic word segmentation information and position information according to the present invention.

### DETAILED DESCRIPTION OF THE EMBODIMENTS

To make the objectives, technical solutions, and advantages of the embodiments of the present application clearer, the following clearly and completely describes the technical solutions in the embodiments of the present application with reference to the accompanying drawings in the embodiments of the present application. Apparently, the described embodiments are some but not all of the embodiments of the present application. Components of the embodiments of the present application described and illustrated in the accompanying drawings can be arranged and designed in various different configurations. Therefore, the detailed description of the embodiments of the present application provided in the accompanying drawings is not intended to limit the scope of the claimed application, but only to represent selected embodiments of the present application. All other embodiments obtained by those ordinary skilled in the art based on the embodiments of the present application without creative efforts shall fall within the protection scope of the present application. Some embodiments of the present application are described in detail below, in conjunction with the accompanying drawings. The following various embodiments and various features in the embodiments can be combined with each other in case of no conflict.

### Embodiments

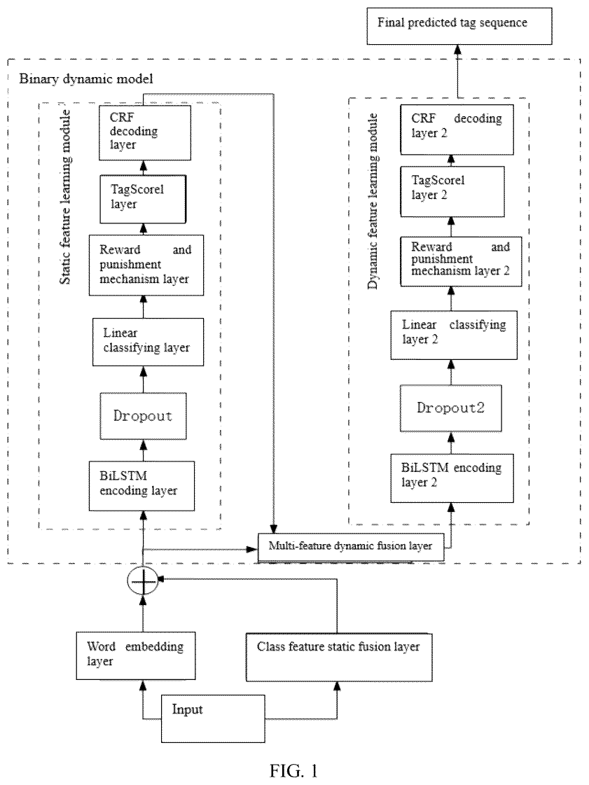

Referring to FIG. 1, a joint extraction system for an entity relationship in the field of traditional Tibetan medicines includes a word embedding layer, a class feature static fusion layer and a binary dynamic model,
where the word embedding layer is used for converting inputted texts into word vectors;
the class feature static fusion layer is used for dividing the inputted texts into three classes of medicinal materials, prescriptions and diagnosis and treatment methods, and fusing the word vectors with the corresponding classes to obtain static fusion features; and
the binary dynamic model is used for acquiring dynamic features according to the static fusion features, fusing the dynamic features with the static fusion features to obtain overall fusion features, and constructing a final predicted tag sequence according to the overall fusion features.

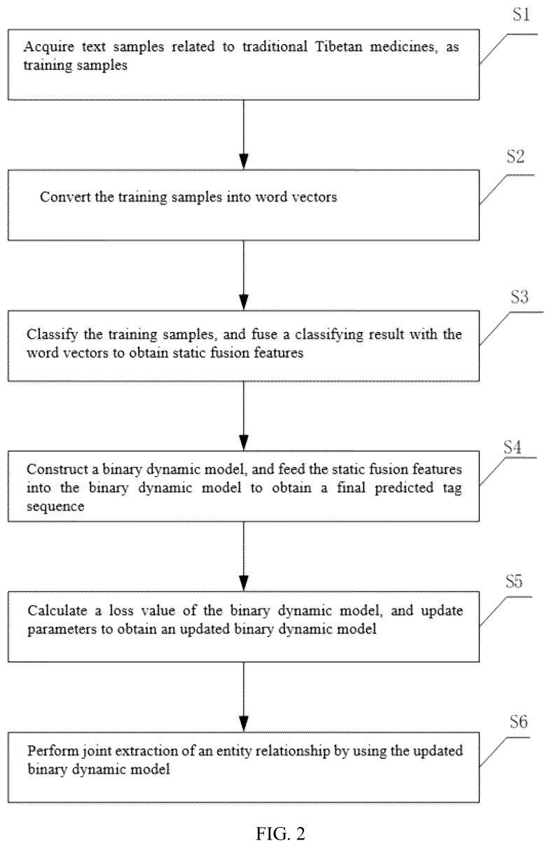

Based on the same inventive concept, referring to FIG. 2, the present invention further provides a joint extraction method for an entity relationship in the field of traditional Tibetan medicines, including the following steps:
S1, text samples related to traditional Tibetan medicines are acquired as training samples;
S2, the training samples are converted into word vectors recorded as (bs, seq_len, dim1), where bs is a batch size, seq_len is a sentence length, and dim1 is a word vector feature dimension;
demonstratively, dimensions of Input are a batch size (batch_size)*a sentence length (seq_len), InputEmbedding is constructed through a vocabulary table size (vocab_size) and dimensions (dimension), input vectors constructed through InputEmbedding are changed into three-dimensional vectors from two-dimensional vectors, dimensions are (batch_size, seq_len, dimension), and at this time, the input vectors can be used as input features of a BiLSTM model and can be encoded; and the input data is labeled through the present application, finally, the input vectors are obtained through an InputEmbedding module, and the nested entities can be labeled.
S3, the training samples are classified, and a classifying result is fused with the word vectors to obtain static fusion features;
the above step S3 includes the following steps:
S3-1, the training samples are classified into three classes of medicinal materials, prescriptions and the diagnosis and treatment methods, to obtain a classifying result as class features of the training samples;
S3-2, each data in the training samples is vectored according to the class features of the training samples to obtain vectored sample data recorded as (bs2, seq_len2, dim2), where bs2 is a batch size of the vectored sample data, seq_len2 is a sentence length of the vectored sample data, and dim2 is a class feature dimension of the vectored sample data; and
S3-3, the vectored sample data is fused with the word vectors to obtain static fusion features recorded as (bs3, seq_len3, dim1+dim2), where bs3 is a batch size of the static fusion features, seq_len3 is a sentence length of the static fusion features, and dim1+dim2 is a fusion feature dimension of the static fusion features.

Demonstratively, the class information is divided into three classes: the medicinal materials, the prescriptions and the diagnosis and treatment methods, and the effects of the models are improved by fusing the class features. vocabsize in an Embedding vectoring method is set as 3, “0” represents the classes of the medicinal materials, “1” represents the classes of the prescriptions, and “2” represents the classes of the diagnosis and treatment methods. Input Embedding and Class Embedding have been obtained through Input, but both are obtained through original Input, without relating to other additional information, and thus, a fusion mode of Input Embedding and Class Embedding is statistic fusion. A symbol “⊕” in FIG. 1 represents the fusion manner of Input Embedding and Class Embedding, but not simple feature addition, first two dimensions are the same as Input Embedding and Class Embedding, but a difference is that the last dimension becomes a sum of two dimensions. A static fusion method of class features is expansion of the feature dimensions, the information of the current features is enriched, and assistance is provided for the BiLSTM model to learn more effective and important information.

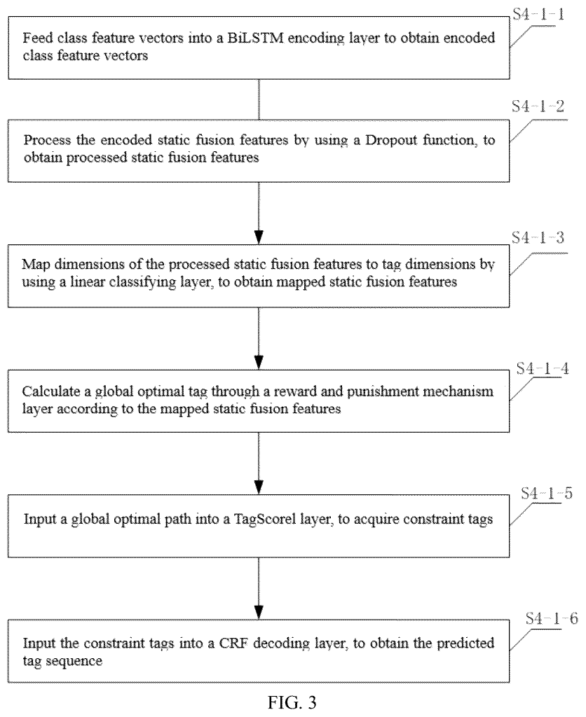

As shown in FIG. 3, in S4, a binary dynamic model is constructed, and the static fusion features are fed into the binary dynamic model to obtain a final predicted tag sequence;
the above step S4 includes the following steps:
S4-1, the static fusion features are fed into a static feature learning module to obtain a predicted tag sequence;
the above step S4-1 includes the following steps:
S4-1-1, the static fusion features are fed into a BiLSTM encoding layer to obtain encoded static fusion features;
in the above steps, the input features are fusion features obtained through the class feature static fusion layer, the features are combined with a two-way semantic learning mechanism of the BiLSTM model, value information in the fusion features can be well dug out, and the foundation is laid for obtaining high-quality word segmentation features (SegmentEmbedding) and high-quality position features (PositionEmbedding) subsequently through an encoding result of the BiLSTM model.
S4-1-2, the encoded static fusion features are processed by using a Dropout function, to obtain processed static fusion features;
in the above steps, use of the Dropout function can avoid over fitting and improve the accuracy of subsequent recognition.
S4-1-3, dimensions of the processed static fusion features are mapped to tag dimensions by using a linear classifying layer, to obtain mapped static fusion features;
in the above steps, the encoding result of the BiLSTM encoding layer is processed through the Dropout function, such that the over fitting problem is avoided.
S4-1-4, a global optimal tag is calculated through a reward and punishment mechanism layer according to the mapped static fusion features;
the above step S4-1-4 includes the following steps:
S4-1-4-1, scores of each path of the training samples are calculated according to the obtained mapped static fusion features;
S4-1-4-2, a loss value loss of a CRF reward and punishment mechanism layer is obtained according to a formula:

$$P ( y \vert x ) = \frac{{e}^{S ( x , {y}^{′} )}}{{\sum }_{i}^{n} {e}^{S ( x , {y}_{i} )}}$$

$$loss = - log ( P ( y \vert x ) ) = log ( {\sum }_{i}^{n} {e}^{S ( x , {y}_{i} )} ) - S ( x , {y}^{′} )$$

where P(y|x) represents a probability that the current path is a correct path, e^{s(x,y′)} represents scores of the current correct path,

$${\sum }_{i}^{n} {e}^{S ( x , {y}_{i} )}$$

represents a sum of possible scores of the each current path, i represents an i^{th} path, n represents n paths in total, S(x,y′) represents scores obtained by correct paths, y′ represents that the current path is the correct path, and x represents an entity;
S4-1-4-3, the reward and punishment mechanism is set, counter propagation is performed by using the loss value loss of the CRF decoding layer, and parameters of the CRF reward and punishment mechanism layer are modified to obtain a modified CRF reward and punishment mechanism layer; and
S4-1-4-4, the global optimal path is calculated by using the modified CRF reward and punishment mechanism layer.

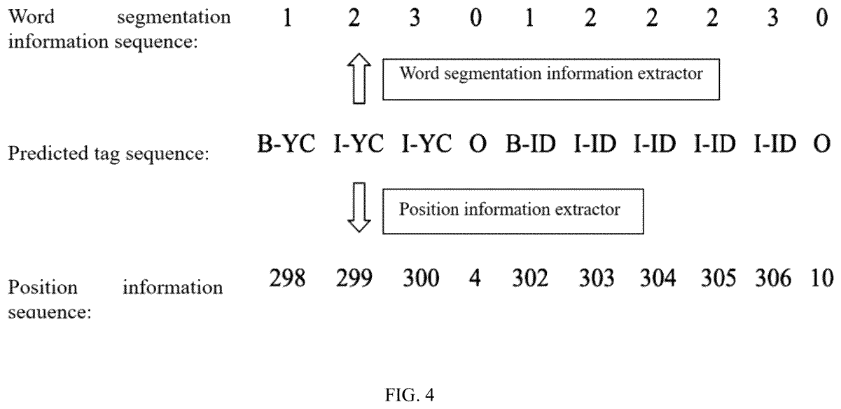

Demonstratively, CRF first calculates the score of each path, and then selects the path with the highest score as a final path. In this process, the scores of different paths are calculated through emissionscore and transitionscore. emissionscore is from a BiLSTM encoding result after being processed through a linear classifying layer Classifier, and contains probability that a current character is predicted to be various tags; and transitionscore is from probability between different tags of different characters, assuming that the current character corresponds to a tag a, a next character corresponds to a tag b, and transitionscore represents the probability between the tag a and the tag b.
S4-1-5, a global optimal path is inputted into a TagScorel layer, to acquire constraint tags; and
S4-1-6, the constraint tags are inputted into a CRF decoding layer, to obtain the predicted tag sequence.
S4-2, the predicted tag sequence and the static fusion features are fed into a multi-feature dynamic fusion layer to obtain overall fusion features; and
as shown in FIG. 4, the step S4-2 includes the following steps:
S4-2-1, the predicted tag sequence is fed into a word segmentation information extractor, and word segmentation information for an entity is constructed according to predicted tags, where the word segmentation information at a start position of the entity is labeled as 1, the word segmentation information at an end position of the entity is labeled as 3, the word segmentation information at a middle position of the entity is labeled as 2, and the word segmentation information of a non-entity is labeled as 0;
S4-2-2, the predicted tag sequence is fed into a position information extractor, and position information for the entity is constructed according to tags;
S4-2-3, the word segmentation information is fused with the position information, to obtain dynamic fusion features; and
S4-2-4, the dynamic fusion features are fused with the static fusion features to obtain the overall fusion features.

Demonstratively, the multi-feature dynamic fusion method has a main objective of enhancing entity boundaries, word segmentation features and position features are dynamically obtained through the predicted tag sequence, the word segmentation features and the position features mainly target named entities, that is, the word segmentation features and the position features for entities are constructed, the entity boundaries in labeled sequences are enhanced through the word segmentation features and the position features, then word segmentation features and the position features are constructed as feature vectors, and then the features are fused with the features obtained by the static fusion method of the class features, such that the quality of the features is improved.

In FIG. 2, the sequences are fed into the word segmentation information extractor, the word segmentation information at a start position of the entity is labeled as 1, the word segmentation information at an end position of the entity is labeled as 3, the word segmentation information at a middle position of the entity is labeled as 2, the word segmentation information of a non-entity is labeled as 0, and the word segmentation information dynamically obtained according to the tag sequences can be used for enhancing the boundary features of the entities in subsequent operations.

The tag sequences are inputted into a position information extractor, first the position information of sentences is constructed according to a length of the tag sequences, and an initial position information sequence of “1 2 3 . . . 10” is constructed. Then, for the tags corresponding to the entities in the sentences, the corresponding positions of the initial position information sequence are summed with the maximum sentence length in a dataset. For example, the maximum sentence length in a traditional Tibetan medicine dataset is 297, the first three Tibetan characters in the sentence form the entity, and thus, the position information is modified to “298 299 300”.
S4-3, the overall fusion features are fed into a dynamic feature learning module to obtain a final predicted tag sequence.

The dynamic feature learning module and the static feature learning module in the step S4-3 have the same structure.

The BiLSTMModel2 encoding layer of the dynamic feature learning module takes the features constructed by the multi-feature dynamic fusion layer as input, and the output of the BiLSTMModel2 encoding layer will be used for constructing the final predicted tag sequence. The binary dynamic model sets two relatively independent encoding modules, which work together to complete joint extraction.
S5, a loss value of the binary dynamic model is calculated, and parameters are updated to obtain an updated binary dynamic model; and
the loss value of the binary dynamic model is calculated in the above step S5 by using a loss value between the predicted tag sequence of the static feature learning module and the final predicted tag sequence of the dynamic feature learning module.

Demonstratively, a gap between the predicted results of a static feature learning structure and a dynamic feature learning structure is reduced, tightness of cooperation between the two structures is enhanced to a certain extent, and a more accurate predicted tag sequence is ultimately outputted.
S6, joint extraction of an entity relationship is performed using the updated binary dynamic model.

In summary, according to the joint extraction system and method for an entity relationship in the field of traditional Tibetan medicines provided by the embodiments of the present application, nested entities can be labeled, a tag classifying result of models can be modified, and prediction accuracy can be improved; and the entity boundaries are enhanced by using dynamic feature fusion, such that the models can better recognize the entities and relationship, and robustness and effectiveness of a system are improved.

It will be apparent to those skilled in the art that the present application is not limited to the details of the above exemplary embodiments, and that the present application can be embodied in other specific forms without departing from the spirit or essential characteristics of the present application. Therefore, from any perspective, the embodiments should be regarded as exemplary and non-restrictive, and the scope of the present application is limited by the accompanying claims rather than the above description. Therefore, it is intended to encompass all variations that fall within the meaning and scope of equivalent elements of the claims within the present application. Any reference numeral in the accompanying drawings in the claims should not be regarded as limiting the claims involved.

What is claimed is:

1. A joint extraction system for an entity relationship in the field of traditional Tibetan medicines, comprising a word embedding layer, a class feature static fusion layer, and a binary dynamic model,
wherein the word embedding layer is used for converting inputted texts into word vectors;
the class feature static fusion layer is used for dividing the inputted texts into three classes of medicinal materials, prescriptions and diagnosis and treatment methods, and fusing the word vectors with the corresponding classes to obtain static fusion features; and
the binary dynamic model is used for acquiring dynamic features according to the static fusion features, fusing the dynamic features with the static fusion features to obtain overall fusion features, and constructing a final predicted tag sequence according to the overall fusion features.

2. A joint extraction method for an entity relationship in the field of traditional Tibetan medicines, applied to the joint extraction system for an entity relationship in the field of traditional Tibetan medicine according to claim 1, comprising the following steps:
S1, acquiring text samples related to traditional Tibetan medicines, as training samples;
S2, converting the training samples into word vectors recorded as (bs, seq_len, dim1), wherein bs is a batch size, seq_len is a sentence length, and dim1 is a word vector feature dimension;
S3, classifying the training samples, and fusing a classifying result with the word vectors to obtain static fusion features;
S4, constructing a binary dynamic model, and feeding the static fusion features into the binary dynamic model to obtain a final predicted tag sequence;
S5, calculating a loss value of the binary dynamic model, and updating parameters to obtain an updated binary dynamic model; and
S6, performing joint extraction of an entity relationship using the updated binary dynamic model.

3. The joint extraction method for an entity relationship in the field of traditional Tibetan medicines according to claim 2, wherein the step S3 comprises the following steps:
S3-1, classifying the training samples into three classes of medicinal materials, prescriptions and the diagnosis and treatment methods, to obtain a classifying result as class features of the training samples;
S3-2, vectoring each data in the training samples according to the class features of the training samples to obtain vectored sample data recorded as (bs2, seq_len2, dim2), wherein bs2 is a batch size of the vectored sample data, seq_len2 is a sentence length of the vectored sample data, and dim2 is a class feature dimension of the vectored sample data; and
S3-3, fusing the vectored sample data with the word vectors to obtain static fusion features recorded as (bs3, seq_len3, dim1+dim2), wherein bs3 is a batch size of the static fusion features, seq_len3 is a sentence length of the static fusion features, and dim1+dim2 is a fusion feature dimension of the static fusion features.

4. The joint extraction method for an entity relationship in the field of traditional Tibetan medicines according to claim 3, wherein the step S4 comprises the following steps:
S4-1, feeding the static fusion features into a static feature learning module to obtain a predicted tag sequence;
S4-2, feeding the predicted tag sequence and the static fusion features into a multi-feature dynamic fusion layer to obtain overall fusion features; and
S4-3, feeding the overall fusion features into a dynamic feature learning module to obtain a final predicted tag sequence.

5. The joint extraction method for an entity relationship in the field of traditional Tibetan medicines according to claim 4, wherein the step S4-1 comprises the following steps:
S4-1-1, feeding the static fusion features into a BiLSTM encoding layer to obtain encoded static fusion features;
S4-1-2, processing the encoded static fusion features by using a Dropout function, to obtain processed static fusion features;
S4-1-3, mapping dimensions of the processed static fusion features to tag dimensions by using a linear classifying layer, to obtain mapped static fusion features;
S4-1-4, calculating a global optimal tag through a reward and punishment mechanism layer according to the mapped static fusion features;
S4-1-5, inputting a global optimal path into a TagScorel layer, to acquire constraint tags; and
S4-1-6, inputting the constraint tags into a CRF decoding layer, to obtain the predicted tag sequence.

6. The joint extraction method for an entity relationship in the field of traditional Tibetan medicines according to claim 5, wherein the dynamic feature learning module and the static feature learning module in the step S4-3 have the same structure.

7. The joint extraction method for an entity relationship in the field of traditional Tibetan medicines according to claim 5, wherein the step S4-1-4 comprises the following steps:
S4-1-4-1, calculating scores of each path of the training samples according to the obtained mapped static fusion features;
S4-1-4-2, obtaining a loss value loss of a CRF reward and punishment mechanism layer according to a formula:
$$P ( y \vert x ) = \frac{{e}^{S ( x , {y}^{′} )}}{{\sum }_{i}^{n} {e}^{S ( x , {y}_{i} )}}$$
$$loss = - log ( P ( y \vert x ) ) = log ( {\sum }_{i}^{n} {e}^{S ( x , {y}_{i} )} ) - S ( x , {y}^{′} )$$
wherein P(y|x) represents a probability that the current path is a correct path, e^{s(x,y′)} represents scores of the current correct path,
$${\sum }_{i}^{n} {e}^{S ( x , {y}_{i} )}$$
represents a sum of possible scores of the each current path, i represents an i^{th} path, n represents n paths in total, S(x,y′) represents scores obtained by correct paths, y′ represents that the current path is the correct path, and x represents an entity;
S4-1-4-3, setting the reward and punishment mechanism, performing counter propagation by using the loss value loss of the CRF decoding layer, and modifying parameters of the CRF reward and punishment mechanism layer to obtain a modified CRF reward and punishment mechanism layer; and
S4-1-4-4, calculating the global optimal path by using the modified CRF reward and punishment mechanism layer.

8. The joint extraction method for an entity relationship in the field of traditional Tibetan medicines according to claim 5, wherein the step S4-2 comprises the following steps:
S4-2-1, feeding the predicted tag sequence into a word segmentation information extractor, and constructing word segmentation information for an entity according to predicted tags, wherein the word segmentation information at a start position of the entity is labeled as 1, the word segmentation information at an end position of the entity is labeled as 3, the word segmentation information at a middle position of the entity is labeled as 2, and the word segmentation information of a non-entity is labeled as 0;
S4-2-2, feeding the predicted tag sequence into a position information extractor, and constructing position information for the entity according to tags;
S4-2-3, fusing the word segmentation information with the position information, to obtain dynamic fusion features; and
S4-2-4, fusing the dynamic fusion features with the static fusion features to obtain the overall fusion features.

9. The joint extraction method for an entity relationship in the field of traditional Tibetan medicines according to claim 8, wherein the loss value of the binary dynamic model is calculated in the step S5 by using a loss value between the predicted tag sequence of the static feature learning module and the final predicted tag sequence of the dynamic feature learning module.

10. An electronic device, comprising:
a memory, being used for storing one or more programs; and
a processor,
wherein when the one or more programs are executed by the processor, the method according to claim 2 is realized.
